(19) United States
(12) Patent Application Publication
(10) Pub. No.: US 2025/0017894 A1
(43) Pub. Date: Jan. 16, 2025

(54) Formulation for Nasal Delivery of Cannabinoids

(71) Applicant: New Leaf Pharmaceuticals, LLC, Newtown, CT (US)

(72) Inventors: Paul CARPENTER, Newtown, CT (US); Rod DERANEY, Newtown, CT (US)

(21) Appl. No.: 18/745,406

(22) Filed: Jun. 17, 2024

Related U.S. Application Data
(63) Continuation of application No. 17/711,523, filed on Apr. 1, 2022, now abandoned.
Application No. 17/711,523 is a continuation of application No. 16/858,024, filed on Apr. 24, 2020, now abandoned.
(60) Provisional application No. 62/809,948, filed on Feb. 25, 2019.

(51) Int. Cl. A61K 31/352; A61K 9/00; A61K 31/05; A61K 31/165; A61K 33/38; A61K 47/44

(52) U.S. Cl. CPC A61K 31/352; A61K 9/0043; A61K 31/05; A61K 31/165; A61K 33/38; A61K 47/44

(57) ABSTRACT

The present invention provides compositions for delivering cannabinoids to the nasal cavity. The subject invention utilizes a composition comprising one or more cannabinoids and an adjuvant mixture to enhance the delivery of the cannabinoid, benefitting the subject to whom the cannabinoid is delivered.

### CROSS-REFERENCE TO RELATED APPLICATION

This application is a continuation application of U.S. patent application Ser. No. 17/711,523, filed Apr. 1, 2022; which is a continuation Application of U.S. patent application Ser. No. 16/858,024, filed Apr. 24, 2020; which claims the benefit of U.S. Provisional Application Ser. No. 62/809,948, filed Feb. 25, 2019, all of which are hereby incorporated by reference herein in their entirety, including any figures, tables, or drawings.

### BACKGROUND OF THE INVENTION

A cannabinoid is one of a class of diverse chemical compounds that can influence cannabinoid receptors in humans and other mammals. In general, there are three sources of cannabinoids. Endocannabinoids are produced naturally in the body. Phytocannabinoids are produced in plants. Synthetic cannabinoids are produced in a laboratory environment.

One notable source of cannabinoids is Cannabis sativa L. Cannabidiol (CBD) is one of 85 cannabinoids found in C. sativa L. While there is a rich history of cannabis use for medicinal purposes, a focus on CBD has not arisen until recently; it has now become known as the main, non-psychoactive cannabinoid found within Cannabis sativa L. Cannabinoids are cyclic molecules exhibiting useful properties such as the ability to easily cross the blood-brain barrier. These compounds also have low toxicity with few side effects. Medicinally, CBD may be used to treat such diverse conditions as anxiety, depression, acne, inflammation, Alzheimer's disease, and Parkinson's disease, making CBD a highly sought-after health-promoting compound.

While CBD is typically administered orally, evidence suggests the oral bioavailability is only 6-15%. Another common method of administering CBD is inhalation using vaporized CBD, which results in 26-36%. Sublingual administration results in 20-35% bioavailability. Intranasal administration is the most effective method of administering CBD. The bioavailability for intranasal administration is generally 34-46%.

Cannabinoids are soluble in highly non-polar solvents (i.e., in substances such as chloroform, dichloromethane and high concentrations of alcohol); they also have limited solubility in glycols. Some of these solvents are pharmaceutically unacceptable, and the pharmaceutically acceptable solvents need to be used in high concentrations to produce solutions which can be applied to the nasal mucosae. Solubility in some of these solvents imposes a ceiling on the dose which can be given using conventional pharmaceutical methods of formulation.

There is a need for safe, natural, and fast acting means of administration of CBD and other cannabinoids by increasing the bioavailability compared to current forms of administration. Preferably, the alternative form of administration should be convenient and discreet.

### BRIEF DESCRIPTION

The subject invention provides compositions and methods of use for nasal delivery of cannabinoids. Advantageously, the present invention provides a composition having all natural, and mostly organic, ingredients that create an adjuvant that facilitates effective delivery of a cannabinoid via the nasal passage.

In the preferred embodiments, the adjuvant composition comprises silver and one or more plant extracts that increase the bioavailability of the cannabinoid.

### BRIEF DESCRIPTION OF THE DRAWING

FIG. 1 is a flowchart of an effective procedure of the subject invention for manufacturing cannabinoid-based health-promoting compound for nasal delivery to a subject.

### DETAILED DISCLOSURE

The subject invention provides compositions and methods of use for nasal delivery of cannabinoids. Advantageously, the present invention provides a composition having all natural, and mostly organic, ingredients that create an adjuvant that facilitates effective delivery of a cannabinoid via the nasal passage.

In the preferred embodiments, the adjuvant composition comprises silver and one or more plant extracts that increase the bioavailability of the cannabinoid.

### Selected Definitions

Cannabinoids are chemicals that bind to a cannabinoid receptor in a subject. Cannabinoids are produced in animals, plants, and synthetically. Over 100 cannabinoids have been identified. As used herein, the term “cannabinoid,” includes, for example, delta-8-tetrahydrocannabinol, delta-9-tetrahydrocannabinol, cannabidiol, olivetol, cannabinol, cannabigerol, nabilone, delta-9-tetrahydro cannabinotic acid, and synthetic analogues thereof as well as the prodrugs and pharmaceutically acceptable salts of cannabinoids.

The term “Cannabis plant(s)” includes wild-type Cannabis sativa and variants thereof, including Cannabis chemovars, which naturally contain different amounts of the individual cannabinoids and also plants that are the result of genetic crosses, self-crosses, or hybrids thereof.

As used herein, the term “adjuvant composition” means a composition that can aid in, contribute to, and/or enhance the effectiveness of a substance that is administered with the adjuvant composition. For example, an adjuvant composition can be included in a prescription drug formulation or a supplement to aid in the effectiveness of the active, primary ingredient(s) for whatever the purpose may be (e.g., treating a disease or enhancing the functioning of an organ or system in the body).

A plant “extract,” as used herein, refers to material resulting from exposing a plant part to a solvent, or from using various chemical, immunological, biochemical or mechanical procedures known to those of skill in the art, including but not limited to, precipitation, steam distillation, centrifugation, filtering, column chromatography, detergent lysis and cold pressing (or expression). Plant extracts can include, for example, essential oils. Plant material from which extracts can be prepared include, for example, roots, stems, leaves, flowers, or parts thereof.

The terms “isolated” or “purified,” when used in connection with biological or natural materials such as polynucleotides, polypeptides, proteins, or other organic compounds, such as small molecules, means that the material is substantially free of other compounds, such as cellular material, with which it is associated in nature. That is, the material does not occur naturally without these other compounds and/or has different or distinctive characteristics compared with those found in the native material.

In certain embodiments, purified compounds are at least 60% by weight of the compound of interest. Preferably, the preparation is at least 75%, more preferably at least 90%, and most preferably at least 99% or 100% (w/w) of the desired compound by weight. Purity is measured by any appropriate standard method, for example, by column chromatography, thin layer chromatography, or high-performance liquid chromatography (HPLC) analysis.

As used herein, the term “subject” refers to an animal, needing or desiring delivery of the benefits provided by a cannabinoid compound. The animal may be, for example, a human, pig, horse, goat, cat, mouse, rat, dog, ape, fish, chimpanzee, orangutan, guinea pig, hamster, cow, sheep, bird, or any other vertebrate or invertebrate. These benefits can include, but are not limited to, the treatment of a health condition, disease, or disorder; prevention of a health condition, disease, or disorder; improve immune health; and/or enhancement of the function of an organ, tissue, or system in the body. The preferred subject, in the context of this invention, is a human. In some embodiments, a subject is suffering from a health condition, disease, or disorder; while, in some embodiments, the subject is in a state of good health (i.e., free from injury or illness) but desires enhanced health and/or functioning of a particular organ, tissue, or body system. The subject can be of any age or stage of development, including infant, toddler, adolescent, teenager, adult, or senior.

The term “pharmaceutically acceptable” as used herein means compatible with the other ingredients of a cannabinoid and/or an adjuvant composition and not harmful to the recipient thereof.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system for which enhancement in health or function is desired; the weight, height, age, and health status of the patient; and the route of administration.

As used herein, the term “treatment” refers to eradicating, reducing, ameliorating, or reversing a sign or symptom of a health condition, disease, or disorder to any extent and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required to, be absolute or complete; meaning, the sign or symptom may still develop at a later time. Prevention can include reducing the severity of the onset of such a condition, disease, or disorder and/or inhibiting the progression of the condition, disease, or disorder to a more severe condition, disease, or disorder.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers, such as 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction or 50 to 40, 50 to 30, 50 to 20, and 50 to 10 in the other direction.

By “reference” is meant a standard or control condition.

By “reduces” is meant as a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “thickener” or “thickening agent” is a substance than can increase the viscosity of a composition. A thickener can be, for example, protein-based or sugar-based. Thickeners are typically isolated from plants, microbes, and animals and are artificially created. Examples include gelatin, cornstarch, xanthan gum, egg whites, and agar.

As used herein, “surfactant” refers to a surface-active substance that lowers the surface tension (or interfacial tension) between two liquids or between a liquid and a solid. Surfactants act as, for example, detergents, wetting agents, emulsifiers, foaming agents, and/or dispersants.

The transitional term “comprising,” which is synonymous with “including,” or “containing,” is inclusive or open-ended and does not exclude additional elements or method steps not recited. By contrast, the transitional phrase “consisting of” excludes any element, step, or ingredient not specified in the claim. The transitional phrase, “consisting essentially of,” limits the scope of a claim to the specified materials or steps “and those that do not materially affect the basic and novel characteristic(s)” of the claimed invention, e.g., the ability to improve bioavailability of cannabidiol. Use of the term “comprising” contemplates other embodiments that “consist” or “consist essentially of” the recited component(s).

Unless specifically stated or is obvious from context, as used herein, the term “or” is understood to be inclusive. Unless specifically stated or is obvious from context, as used herein, the terms “a,” “an,” and “the” are understood to be singular or plural.

Unless specifically stated or is obvious from context, as used herein, the term “about” is understood as within a range of normal tolerance in the art, for example, within 2 standard deviations of the mean. The term “about” can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof and from the claims. All references cited herein are hereby incorporated by reference in their entirety.

### Formulation and Delivery of a Cannabinoid

The subject invention facilitates effective absorption of cannabinoids through the nasal passage of the subject, preferably as a nasal spray; however, additional forms of nasal absorption are contemplated.

Advantageously, the present invention provides a nasal spray using all natural, and mostly organic, ingredients. The materials used in the present invention are specifically designed to provide a safe, non-addictive nasal spray having a high concentration of medicinal cannabinoid. The formulation described herein is designed to be an over-the-counter homeopathic drug with a National Drug Code (NDC). In additional embodiments, the formulation may also be distributed as a prescription composition.

In accordance with a preferred embodiment of the subject invention, a cannabinoid is formulated with an adjuvant composition. The adjuvant composition comprises a silver solution and a plant extract. In certain embodiments, the cannabinoid is mixed with all of the components of the adjuvant composition simultaneously. In other embodiments, the cannabinoid is mixed sequentially with the various components of the adjuvant mixture.

In some embodiments, one cannabinoid or a combination of cannabinoids, are mixed with water, preferably USP water. The cannabinoid is added to achieve a concentration in the solution of about 0.01% to about 50%, about 0.025% to about 25%, or about 0.05% to about 10%.

In one embodiment, the cannabinoid(s) is/are isolated from a Cannabis plant, preferably Cannabis sativa L. Cannabinoid contains a broad-spectrum of molecules, preferably comprising cannabigerol (CBG), cannabinol (CBN), cannabichromene (CBC), and/or cannabidiol (CBD). The isolated cannabinoids can also, optionally, include cannabidivarin, tetrahydrocannabicarin, cannabichromene, cannabidiolic acid, cannabinolic aicd, and/or cannabichromenic acid.

In certain embodiments, the cannabinoid solution of the subject invention also contains one or more surfactants. The surfactant component can be combined with the cannabinoid at a ratio of about 10,000:1 to about 1:1, about 5000:1 to about 100:1, about 2500:1 to about 500:1, or about 1000:1 (cannabinoid(s):surfactant). In some embodiments, the surfactant is an emulsifier. In certain embodiments, the emulsifier is a fatty acid. In some embodiments, the surfactant is derived from natural sources such as eukaryotes, including plants, fungi, or animals; or, it is obtained from bacteria. In certain embodiments, the surfactant is created synthetically.

In some embodiments, the adjuvant mixture comprises a silver solution and one or more plant extracts. One example of a silver-based solution is SilverSol®, supplied by Silver Biotics®, which is a mixture of silver and deionized water. In some embodiments, the silver solution has a concentration of silver of about 0.0001 to about 10%, about 0.0005% to about 1%, or about 0.001% to about 0.1%. The silver solution is added to the cannabinoid composition to achieve a concentration of the silver solution of about 0.01% to about 50%, about 5% to about 25%, or about 7.5% to about 15%.

In some embodiments, the plant extract is a capsaicinoid. In certain embodiments, the capsaicinoid is capsaicin. The capsaicinoid may be purified before the addition to the adjuvant composition. In one embodiment it is added in conjunction with a pharmaceutically acceptable solution designed for intranasal administration, preferably Sinol-M™ Headache, a product supplied by Sinol USA, Inc. Sinol-M™ Headache comprises of Aloe vera extract, rosemary extract, sea salt, vitamin C, and capsicum. The capsaicinoid is added to achieve a concentration in the composition of about 0.00001% to about 1%, about 0.0001% to about 0.1%, or about 0.0005% to about 0.01%.

The pharmaceutically acceptable solution for intranasal administration may have a variety of carriers, excipients, plants extracts, essential oils, and/or salts.

The nasally-administered composition can further comprise one or more essential oils, botanicals, or other plant extracts comprising compounds with therapeutic antiviral, antibacterial, anti-biofilm, anti-inflammatory, and/or immunomodulatory properties, such as, for example, terpenes and/or phenols. These can include horseheal (Inula helenium, L. Asteraceae, elecampane), rose (Rosa damascena L., Rosaceae), lavender (Lavandula angustifolia L., Labiatae), chamomile (Matricaria recutica L., Asteraceae), orange (Rutaceae), grapefruit (Citrus paradisi), eucalyptus (Eucalyptus globulus L., Myrtaceae), geranium (Geranium robertianum L., Geraniaceae), juniper (Juniperus communis L., Cupressaceae), citrus (Citrus sinensis L., Rutaceae), tea tree (Melaceuca alternifolia), manuka bush (Leptospermum scoparium), neem tree (Azadirachta indica, A. Juss), tea plant (Camellia sinensis), rosemary (Rosmarinus officinalis L., Lamiaceae), eucalyptus (e.g., Eucalyptus globulus), clove (Syzygium aromaticum), lemon, oregano, cinnamon, citronella, and thyme oils.

In a specific embodiment, the composition comprises about 0.001% to about 10%, about 0.01% to about 1%, or about 0.025% to about 0.1% of the majority component of eucalyptus oil-eucalyptol.

In certain embodiments, a thickening agent is mixed into the composition at a concentration of about 0.01% to about 1%, about 0.05% to about 0.5%, or about 0.075% to about 0.25%. The thickening agent is, preferably, a polysaccharide. More preferably, the thickening agent is a gum, such as, but not limited to, xanthum gum, cellulose gum, guar gum, or locust bean gum.

In certain embodiments, the final cannabinoid composition is homogenized. For example, the composition can be mixed for about 1 minute to about 240 minutes, about 5 minutes to about 60 minutes, or about 10 minutes to about 30 minutes at an rpm of about 500 to about 20000, about 2000 to about 17500, or about 8000 to about 15000. Nano-technology can also be used to further reduce the molecule size for even better absorption.

In some embodiments, the cannabinoid composition is mixed with various components of the adjuvant mixture progressively. After any one or a combination of components that makeup the adjuvant composition is added to the cannabinoid, the composition can be homogenized. The composition can be homogenized for about 1 minute to about 240 minutes, about 2 minutes to about 60 minutes, or about 3 minutes to about 10 minutes at an rpm of about 500 to about 15000, about 2000 to about 12000, or about 3000 to about 9000.

The composition may have from 0.1% to about 99% (by weight or by volume) of one or more additional components including, for example, carriers, pH modifiers, buffers, chelators, local anesthetic agents, agents that promote wound healing, agents that help degrade biofilm, agents that stop bleeding and/or promote clot formation, carriers, and other therapeutic and non-therapeutic components, such as, for example, anti-viral agents, fungicidal agents, chemotherapeutic agents, topical antiseptics, anesthetic agents, oxygenated fluids and/or agents, diagnostic agents, homeopathic agents, and over-the-counter medications/agents.

In certain embodiments, the composition comprises a smoothing agent, such as glycerol or glycerin. In certain embodiments, the composition comprises an emulsifier, such as a salt selected from, for example, sodium chloride, sodium lactate, calcium lactate, sodium citrates, potassium citrates, calcium citrates, sodium phosphates, potassium phosphates, diphosphates, triphosphates, polyphosphates, and tartrates.

Carriers and/or excipients according the present invention can include any and all solvents, diluents, buffers (e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Tween 80, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners, coatings, preservatives (e.g., Thimerosal, benzyl alcohol), antioxidants (e.g., ascorbic acid, sodium metabisulfite), tonicity controlling agents, absorption delaying agents, adjuvants, bulking agents (e.g., lactose, mannitol), and the like.

In some cases, the carriers can be, for example, sterile or non-sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other known carriers appropriate to specific routes of administration. The use of carriers and/or excipients in the field of drugs and supplements is well known. Except for any conventional media or agent that is incompatible with the supplement composition or with, its use in the present compositions may be contemplated.

In one embodiment, the composition can be made into an aerosol or spray formulation so that, for example, it can be nebulized or inhaled. Suitable pharmaceutical formulations for administration in the form of aerosols or sprays are, for example, solutions, suspensions, or emulsions. Formulations for nasal aerosol or inhalation administration may also be formulated with illustrative carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a metered dose nasal spray, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

Further components can be added to the compositions as are determined by the skilled artisan, for example, buffers, carriers, viscosity modifiers, preservatives, flavorings, dyes, and other ingredients specific for an intended use. One skilled in this art will recognize that the above description is illustrative rather than exhaustive. Indeed, many additional formulations techniques and pharmaceutically-acceptable excipients and carrier solutions suitable for particular modes of administration are well-known to those skilled in the art.

### Example

A greater understanding of the present invention and of its many advantages may be had from the following example, given by way of illustration. The following example is illustrative of some of the embodiments, variants, methods, and applications of the present invention. It is not to be considered as limiting the invention.

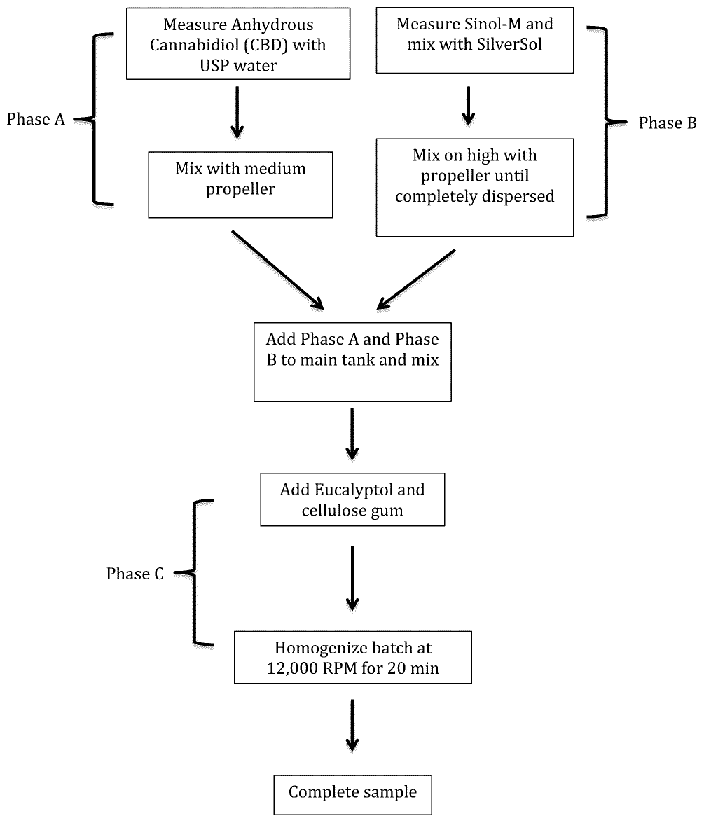

With reference now to FIG. 1, an example of the production of a cannabinoid composition for intranasal administration is illustrated. Phase A is shown to include water and a broad-spectrum cannabinoid composition that comprises CBD, CBN, and CPG. Generally, 82.92 mL of USP water is mixed with 0.95 g of CBD with fatty acids at a ratio of 1000:1 (cannabinoids:fatty acids), enabling the emulsification of the insoluble cannabinoids into USP water. The Phase A solution is then mixed with a medium propeller.

Continuing with FIG. 1, in Phase B approximately 4 g of Sinol-M™ Headache is mixed at an rpm of 4000 with 12 g of SilverSol®. Next, Phase A is added to a main tank with Phase B and mixed at 8000 rpm for 5 minutes.

In Phase C, shown in FIG. 1, approximately 0.05 g of eucalyptol is mixed until it has completely dispersed into the solution of the cannabinoids, Sinol-M™ Headache, and SilverSol®. The batch is then homogenized on 12,000 rpm for 20 minutes. The sample is complete and available for testing or dispensing into a nasal spray dispenser.

It is envisioned that the product formed by mixing these ingredients is dispensed into a vial and closed with a pump action spray button. A metered dose spray or pump is used to achieve the exact measurement of the cannabinoids into the nasal mucosa with each use.

We claim:

1. A composition for application in the nasal cavity of a subject, the composition comprising:
a) a cannabinoid;
b) silver; and
c) a plant extract.

2. The composition of claim 1, wherein the cannabinoid comprises one or a combination of the following cannabinoid chemicals: cannabidiol (CBD), cannabigerol (CBG), cannabichromene (CBC), or cannabinol (CBN).

3. The composition of claim 1, wherein the cannabinoid is present at about 0.1% to about 10% in the composition.

4. The composition of claim 1, wherein the silver is present at a concentration of about 0.000001% to about 1% in the composition.

5. The composition of claim 2, wherein the cannabinoid is present at a concentration of at least 2%.

6. The composition of claim 1, wherein the plant extract is present at a concentration of about 0.0001% to about 10% in the composition.

7. The composition of claim 1, wherein the plant extract is an essential oil.

8. The composition of claim 7, wherein the essential oil is eucalyptol.

9. The composition of claim 1, wherein the plant extract is capsaicin.

10. The composition of claim 1, further comprising a pharmaceutically acceptable carrier.

11. The composition of claim 1, further comprising a surfactant.

12. The composition of claim 11, wherein the surfactant is a fatty acid.

13. A method for delivering a cannabinoid to a subject, wherein said method comprises administering to the nasal cavity of the subject a composition of claim 1.
